(19) United States
(12) Patent Application Publication
(10) Pub. No.: US 2020/0230059 A1
(43) Pub. Date: Jul. 23, 2020

(54) Solid Micronized Melatonin Composition

(71) Applicant: Physician's Seal, LLC, Boca Raton, FL (US)

(72) Inventors: Syed M. Shah, Boca Raton, FL (US); Daniel Hassan, Boca Raton, FL (US)

(21) Appl. No.: 16/746,451

(22) Filed: Jan. 17, 2020

Related U.S. Application Data
(60) Provisional application No. 62/794,159, filed on Jan. 18, 2019.

(51) Int. Cl. A61K 9/14; A61K 9/50; A61K 47/38; A61K 47/32; A61K 9/20; A61K 47/12; A61K 31/4045

(52) U.S. Cl. CPC A61K 9/14; A61K 9/5089; A61K 47/38; A61K 31/4045; A61K 9/2095; A61K 47/12; A61K 47/32

(57) ABSTRACT

A melatonin composition has a dry granulation including a melatonin powder with a median melatonin particle size of 5 μm to 40 μm, a carboxylic acid powder, and a powder of a hydrogel-forming polymer. The dry granulation is combined with pharmaceutical excipients in a dry orally ingestible pharmaceutical dosage form adapted to absorb water upon ingestion and form a hydrogel including soluble melatonin and soluble carboxylic acid in the hydrogel, the carboxylic acid being in an amount sufficient to impart a pH of 4.4 or less to the hydrogel after ingestion.

### CROSS-REFERENCE TO RELATED APPLICATION

This claims the benefit of priority from U.S. provisional Application No. 62/794,159, filed Jan. 18, 2019, which is incorporated by reference in its entirety.

### FIELD

This relates to the field of melatonin compositions and, more particularly, to melatonin solid oral dosage forms.

### BACKGROUND

Melatonin is a common dietary supplement that has been shown to be effective at treating circadian rhythm disorders, sleep disorders, jet lag, shift work syndrome, seasonal affective disease, insomnia, melatonin deficiency in the elderly, and many other conditions. It is typically administered in an oral tablet or liquid drop dosage form.

Certain drawbacks to with conventional melatonin products were solved by the formulation described in International Publication No. WO 2012/103411. This publication describes a controlled-release melatonin composition that can be delivered to the gastrointestinal tract and will release a bioavailable amount of melatonin therein the over the course of 6-8 hours. The composition in that publication includes melatonin dispersed in a hydrogel matrix having sufficient acidic moieties therein to maintain an acidic environment local to the melatonin when the melatonin is in the gastrointestinal tract. The melatonin was maintained in a local pH environment in the dosage form that kept it soluble regardless of the pH of the GI tract environment.

### BRIEF SUMMARY

Although the formulation described in International Publication No. WO 2012/103411 is effective, what is needed is a new melatonin composition that has improved processing and performance features.

A method of making such a melatonin dosage form comprises

forming granules by dry granulating a melatonin powder having a median melatonin particle size of 5 μm to 40 μm, a carboxylic acid powder, and a powder of a hydrogel-forming polymer to form a dry granulation having a substantially uniform distribution of melatonin powder, carboxylic acid powder, and powder of the hydrogel-forming polymer. The granules are placed into a dry orally ingestible pharmaceutical dosage form adapted to absorb water upon ingestion and form a hydrogel including soluble melatonin and soluble carboxylic acid in the hydrogel. The carboxylic acid is in an amount sufficient to impart a pH of 4.4 or less to the hydrogel after ingestion.

Additional features of the method may include one or more of the following features.

Dry granulating may be performed without including a liquid solvent.

The dosage form may be selected from a tablet, capsule, caplet, and multiparticulate.

The carboxylic acid may be citric acid.

A particle size of the carboxylic acid may be greater than the particle size of the melatonin.

The method may further comprise compacting the granules by roller compaction and/or slugging after the forming step and prior to the placing step.

The dosage form may comprise 0.4% w/w to 8% w/w melatonin, 12% w/w to 40% w/w carboxylic acid, and 8% w/w to 48% w/w hydrogel-forming polymer.

The dosage form may comprise 0.4% w/w to 8% w/w melatonin, 24% w/w to 30% w/w carboxylic acid, and 16% w/w to 24% w/w hydrogel-forming polymer.

The dosage form may provide a sustained release of melatonin after ingestion for 3-10 hours regardless of the pH environment the dry orally ingestible pharmaceutical dosage form passes through.

The melatonin powder and carboxylic acid powder may be in direct physical contact in the granules.

An example of such a melatonin composition comprises a dry granulation including a melatonin powder with a median melatonin particle size of 5 μm to 40 μm, a carboxylic acid powder, and a powder of a hydrogel-forming polymer. The dry granulation is combined with pharmaceutical excipients in a dry orally ingestible pharmaceutical dosage form. The dosage form is adapted to absorb water upon ingestion and form a hydrogel including soluble melatonin and soluble carboxylic acid in the hydrogel. The carboxylic acid being in an amount sufficient to impart a pH of 4.4 or less to the hydrogel after ingestion.

The dosage form may be at least one of a tablet, capsule, and multiparticulate.

The carboxylic acid may be citric acid.

A particle size of the carboxylic acid may be greater than the particle size of the melatonin.

The dosage form may comprise 0.4% w/w to 8% w/w melatonin, 12% w/w to 40% w/w carboxylic acid, and 8% w/w to 48% w/w hydrogel-forming polymer.

The dosage form may comprise 0.4% w/w to 8% w/w melatonin, 24% w/w to 30% w/w carboxylic acid, and 16% w/w to 24% w/w hydrogel-forming polymer.

The dosage form may provide provides a sustained release of melatonin after ingestion for 3-10 hours regardless of the pH environment the dosage form passes through.

The melatonin powder and carboxylic acid powder may be in direct physical contact in the dry granulation.

A method of treatment comprises administering to a patient in need of melatonin therapy a therapeutically effective amount of a dry orally ingestible pharmaceutical dosage form having therein a dry granulation including a melatonin powder with a median melatonin particle size of 5 μm to 40 μm, a carboxylic acid powder, and a powder of a hydrogel-forming polymer. The dry granulation is combined with pharmaceutical excipients in the dosage form. The dosage form is adapted to absorb water upon ingestion and form a hydrogel including soluble melatonin and soluble carboxylic acid in the hydrogel. The carboxylic acid is in an amount sufficient to impart a pH of 4.4 or less to the hydrogel after ingestion.

The dosage form may be selected from a tablet, capsule, caplet, and multiparticulate.

The carboxylic acid may be citric acid.

A particle size of the carboxylic acid may be greater than the particle size of the melatonin.

The dosage form may comprise 0.4% w/w to 8% w/w melatonin, 12% w/w to 40% w/w carboxylic acid, and 8% w/w to 48% w/w hydrogel-forming polymer.

The dosage form may comprise 0.4% w/w to 8% w/w melatonin, 24% w/w to 30% w/w carboxylic acid, and 16% w/w to 24% w/w hydrogel-forming polymer.

The dosage form may provide a sustained release of melatonin after ingestion for 3-10 hours regardless of the pH environment the dosage form passes through.

The melatonin powder and carboxylic acid powder may be in direct physical contact in the dry granulation.

### DETAILED DESCRIPTION OF EXAMPLE EMBODIMENTS

Melatonin is a difficult active ingredient to deliver in a controlled release dosage form. It has been reported to have a pKa of approximately 4.4 to 4.7, thus it has a different degree of solubility in different parts of the GI tract. In the gastric environment where the pH ranges from approximately 1 to 3, its solubility is relatively high. In the upper GI tract environment where the pH is approximately 4.5 to 5.5, its solubility decreases. In the lower GI tract environment where the pH is approximately 5.5 to 7, its solubility decreases even further. This variability in the pH of the GI tract is not a problem for conventional immediate-release dosage forms, as melatonin is readily dissolved in the low pH of the stomach.

In the melatonin source used in WO 2012/103411, the melatonin source was a solid powder having a predominant particles size of 150 μm. The melatonin powder was dissolved in polyethylene glycol (“PEG”) to put it in a soluble form prior to tablet granulation. The melatonin/PEG solution was sprayed into a wet granulation mixture with dry tablet excipients and dry citric acid, then the wet granulation was dried. The dried granulation had to be milled to achieve ingredient uniformity.

A problem with using large melatonin particles, such as the 150 μm particles used in WO 2012/103411 without dissolving them, is that the large particles are difficult to blend uniformly with the other ingredients in the dosage form. In order to get a more uniform distribution of melatonin in the dosage form, WO 2012/103411 reported dissolving the melatonin in PEG, which is not ideal in all situations.

Obtaining substantially uniform distribution is important for obtaining the desired physiological release kinetics. In the dosage form, the melatonin should be directly in contact with the acid throughout so that when the dosage form is swallowed and absorbs water, the acid can dissolve within the polymer matrix and set up an acidic environment within the matrix, thereby allowing the melatonin to also dissolve. This puts the melatonin in a locally acidic environment where it is water soluble. If the acid and melatonin are spread apart in the dosage form, individual melatonin vesicles can form in the polymer matrix that are not in a locally acidic environment. Because the melatonin in these vesicles is not very soluble, it will not be released from the dosage form as desired.

The dosage form in WO 2012/103411 was prepared using a wet granulation process. The problem with wet granulating melatonin is that melatonin can undergo deamination upon exposure to water and dissolved citric acid and from heat during the drying step. This can lead to content non-uniformity and temperature-related stability issues. Melatonin can also settle in the granulation solution, leading to non-uniform granulations.

It would be advantageous to be able to use solid melatonin in the dosage form rather than a dissolved form of melatonin while also being able to obtain a substantially uniform distribution of melatonin and acid throughout the dosage form, allowing substantially all the melatonin to be in direct physical contact with the acid in the dosage form before it is swallowed. It would also be advantageous to be able to use a dry granulation process to prepare the melatonin dosage form.

The melatonin composition described here improves upon the formulation described WO 2012/103411. It eliminates the need to dissolve melatonin in PEG or another solubilizing agent prior to granulation and eliminates the need to mill the dried granulation to obtain ingredient uniformity. It also unexpectedly provides a faster onset of action or release from the dosage form and more reliably releases its melatonin content throughout the GI tract regardless of the pH variations within the GI tract. Details of the new and advantageous melatonin composition are now described.

The composition includes melatonin as an active ingredient. The source of melatonin is a powder advantageously selected to be substantially pure (at least 99.8% pure melatonin), but also have a very small particle size. The melatonin powder is used directly in the dosage form. It is not dissolved in a solvent prior to preparing the dosage form.

The term “particle size” as used here refers to the size of individual particles making up a powder, which may be polycrystalline. The sizes of individual particles in a powder are not usually uniform; instead they are distributed over a range of sizes, which may vary around a median particle size.

A conventional technique of reporting measurements of particle size to report the D-values D10, D50, and D90 for a powder sample. D10 is the size for which 10% of the sample's mass is particles with a critical dimension less than the value. D50 is the diameter of the particles for which 50% of the sample's mass is smaller than the value and 50% of a sample's mass is larger than the value. D90 is the critical dimension of the particles for which 90% of the sample's mass is smaller than the value and 100% of a sample's mass is larger than the value. A particle size of a powder sample may be measured by sieving, laser diffraction, light scattering, and/or image analysis. The critical dimension refers to one of the dimensions of an individual particle from one side to the other. On a sphere, for example, the critical dimension would be the diameter.

In some examples of the composition, the median particle size of individual melatonin crystals in the powder is in the range of 5 μm to 40 μm. In some examples of the composition, the particle size distribution is D10≤5, D50≤20, and D90≤40.

The composition further includes a carboxylic acid powder. The carboxylic acid powder may be a powder of a low molecular weight carboxylic acid such as citric acid, succinic acid, tartaric acid, or the like. Using a powder form of the carboxylic acid in the dosage form helps ensure a substantially uniform pH within the hydrogel matrix in the gastrointestinal tract, helps prevent formation of channels in the dosage form, which can cause non-uniform release of melatonin due to non-hydrogel containing cavities left after dissolution of larger crystals of the carboxylic acid, allow for improved tablet strength, and provide improved content uniformity of the melatonin, within the dosage from.

The powder form of the carboxylic acid may be obtained from the producer, in a milled or micronized form, or it may be formed by milling larger crystals of the acid by itself or in combination with the other ingredients of the formulation (i.e. excipients, with or without, the melatonin active). It may also be advantageous in some cases to use roller compaction of the dry blend of the acidic moiety along with other ingredients of the formulation, prior to milling, to improve content uniformity, tablet strength and reliability in tablet manufacturing.

The amount of carboxylic acid powder is sufficient to impart an acidic pH to the polymer matrix when it absorbs water. Some suitable pH ranges for the polymer matrix provided by the amount of carboxylic acid powder include 0.1 to 5, 1 to 5, 2 to 5, 2 to 4.5, 3 to 5, 3 to 4.5, 3.3 to 5, or 3.4-4.5, or 4.4 or less.

This may be useful for obtaining a sustained release of melatonin throughout the GI tract. Melatonin is much more soluble in the stomach than the intestines because the pH of the stomach is low whereas the pH of the intestines is higher. The acidified polymer matrix forms a controlled pH carrier for the melatonin within the GI tract. Melatonin remains solubilized in the matrix when it absorbs water in the GI tract and can gradually release from the matrix as the dosage form passes through the GI tract, regardless of the GI tract's local pH environment.

In the dosage form, the melatonin and acid are located within a polymer matrix. The polymer matrix is formed from at least one pharmaceutically acceptable polymeric excipient. Examples of polymeric excipients include, but are not limited to: cellulosic polymers such as carboxymethylcelluloses, methylcelluloses, hydroxypropylcelluloses, and hydroxypropylmethylcelluloses; hyaluronates; alginates; polysaccharides, heteropolysaccharides, pectins; poloxamers; poloxamines; ethylene vinyl acetates; polyethylene glycols; dextrans; polyvinylpyrrolidones; chitosans; polyvinylalcohols; propylene glycols; polyvinylacetates; phosphatidylcholines, lecithins; miglyols; polylactic acid; polyhydroxybutyric acid; mixtures thereof, copolymers thereof, derivatives thereof, and the like.

In a particular example, the polymeric excipient is a hydrogel-forming polymer. A hydrogel-forming polymer is a polymer capable of absorbing water. When the hydrogel-forming polymer forms a hydrogel, the melatonin and carboxylic acid dissolve in the hydrogel and disperse throughout the hydrogel and gradually exit the hydrogel into the GI tract. The hydrogel-forming polymer may act as a release-controlling polymer to provide a sustained release of melatonin into the GI tract over a desired time period. Hydroxypropyl methylcellulose (“HPMC” or “hypromellose”) is used in certain particular formulations of the dosage form because it forms a hydrogel, is safe, and works well with melatonin and the acid.

In the finished dosage form that is ready to be administered to a subject, the micronized melatonin particles are in direct physical contact with the carboxylic acid particles. Because the carboxylic acid particles have a particle size much larger than the melatonin particles, each carboxylic acid particle may be in direct physical contact with multiple melatonin particles. Thus, when the dosage form is swallowed, almost all of the melatonin will be exposed to the acidified polymer matrix and dissolve within the dosage form. In the end, when compared to the formulation of WO 2012/103411, the amount of melatonin in the dosage form that is in soluble form is higher, ensuring a more reliably measurable dose of melatonin is released into the GI tract.

The polymer matrix effectively insulates the melatonin from the pH environment of the GI tract. Instead of dissolving directly into the GI tract, the melatonin dissolves within the acidified polymeric matrix, forming a concentration gradient across the matrix. Melatonin will then be released into the GI tract from the periphery of the matrix in this manner.

The dosage form may be adapted to release an effective amount of melatonin within the pH range found in the intestines continuously for at least 3 and up to 10 hours. In a particular example, the dosage form is adapted to release melatonin over a period of 3-10 hours after ingestion regardless of the pH environment it passes through. This sustained release melatonin will help the subject remain asleep through the night.

As mentioned above, the dosage form will typically be an oral dosage form such as a tablet, caplet, capsule, multiparticulate, or the like. One or more pharmaceutically acceptable excipients aside from those described already may be used to obtain the desired dosage form and give it the desired properties.

Examples of excipients include, but are not limited to, carriers, diluents, disintegrants, emulsifiers, solvents, processing aids, buffering agents, colorants, flavorings, solvents, coating agents, binders, carriers, glidants, lubricants, granulating agents, gelling agents, polishing agents, suspending agent, sweetening agent, anti-adherents, preservatives, emulsifiers, antioxidants, plasticizers, surfactants, viscosity agents, enteric agents, wetting agents, thickening agents, stabilizing agents, solubilizing agents, bioadhesives, film forming agents, emollients, dissolution enhancers, dispersing agents, or combinations thereof.

Conventional processing aids may be used to prepare dosage form. Examples of processing aids include, but are not limited to, magnesium stearate, stearic acid, talc, and sodium lauryl sulfate.

The dosage form may include a pharmaceutically acceptable filler. Examples of fillers include, but are not limited to, silicates, calcium carbonate, glycine, dextrin, sucrose, sorbitol, dicalcium phosphate, calcium sulfate, lactose, kaolin, mannitol, sodium chloride, talc, dry starches and powdered sugar.

The dosage form may include a pharmaceutically acceptable binder. Examples of binders include, but are not limited to, cellulosic, and povidone binders such as microcrystalline cellulose, hydroxypropyl methylcellulose, and crospovidone.

The dosage form may be coated to aid in swallowing, to mask the taste of the ingredients, improve appearance, to protect the dosage form from moisture, and/or to have an enteric coating. The coating may be applied using conventional coating techniques, such as, for example, spray coating, bed coating, or the like.

The dosage form may be coated with an enteric coating to substantially prevent the active ingredients from releasing into the stomach. Examples of enteric coating materials include shellac, cellulose acetate phthalate, polyvinyl acetate phthalate, ethyl cellulose/sodium alginate, hypromellose acetate succinate, or a methacrylic acid-based polymer or copolymer such as methacrylic acid-ethyl acrylate copolymer.

The dosage form may include a sustained release portion, which is the polymer matrix containing the melatonin. The sustained release portion is effective to release the melatonin therefrom into the patient's lower GI tract over about 3 hours to about 10 hours after oral ingestion by the patient. In certain cases, the dosage form will release substantially all of the melatonin therefrom within 10 hours after oral ingestion or within about 8 hours after oral ingestion.

The dosage form may also include an expedited release portion. The expedited release portion is effective to release about 50% of the melatonin into the lower GI tract within about 2 hours after oral ingestion or about 1 hour after oral ingestion.

The expedited release portion of the dosage form can be formulated many different ways. A few examples are described below, but these examples are not an exhaustive list of the many possibilities.

The polymer matrix may function as both the expedited release portion and sustained release portion. This is because when the dosage form reaches the patient's stomach, it will begin releasing some of the melatonin from the polymer matrix almost immediately as the polymer matrix absorbs water in the stomach. As the polymer matrix swells, a pH gradient forms within the matrix and the release rate of the melatonin slows.

Another example of a dosage form with an expedited release portion and sustained release portion is a bi-layer tablet having one layer forming the sustained release portion and another layer forming the expedited release portion.

Another example of a dosage form with an expedited release portion and sustained release portion is a capsule containing the sustained release portion and expedited release portion. In such an example, the expedited release portion may include particulates effective to release the melatonin therein over a desired expedited time period and the sustained release portion may be another set of particulates effective to release the melatonin therein for a sustained time period.

Another example of a dosage form with an expedited release portion and sustained release portion is a tablet or capsule in which the polymer matrix forms a solid core and the expedited release portion is in a coating over the core.

The relative dosage percentage of the expedited and sustained release portions can vary. In some examples, the expedited release portion contains 5% to 50% or up to 65% of the melatonin in the dosage form. In other examples, the sustained release portion contains up to 90% of the melatonin in the dosage form. In another example, the expedited release portion contains approximately 50% of the melatonin.

The active ingredients from the expedited release portion are released approximately in the first two hours after ingestion. The active ingredients in the sustained release portion include the remainder of the active ingredient(s), which are released approximately over the next 5-8 hours or within about 10 hours after ingestion.

The release profile may be measured by simulating the GI tract environment by placing the dosage form in a 0.1 N HCl (hydrochloric acid) solution for two hours, then placing it in a phosphate buffer solution of pH=6.8 for 12 hours.

The release rate of the melatonin from the dosage form can be controlled in several ways. The concentration of the active ingredient(s) may be adjusted. The pH of polymer matrix may be adjusted. One or more release rate controlling coatings may be included. The thickness of such a coating may be adjusted. The size and shape of the dosage form may also be adjusted to provide the preferred release rate.

An effective amount is an amount that is sufficient to provide a therapeutic benefit affecting a disease or condition in the body.

A therapeutically effective amount of melatonin may be 0.1-1,000 mg/day, including 0.1-25 mg/day, 0.1-10 mg/day, 1-20 mg/day, 1-10 mg/day, 2-10 mg/day, 50-75 mg/day, 75-100 mg/day, 100-150 mg/day, 150-200 mg/day, 200-250 mg/day, 250-300 mg/day, 300-350 mg/day, 350-400 mg/day, 400-450 mg/day, 450-500 mg/day, 500-550 mg/day, 550-600 mg/day, 600-650 mg/day, 650-700 mg/day, 700-750 mg/day, 750-800 mg/day, 800-850 mg/day, 850-900 mg/day, 900-950 mg/day, 950-1,000 mg/day. Higher doses (1,000-3,000 mg/day) might also be effective. The weight in mg is often calibrated to the body weight of the patient in kg, thus these example doses may also be written in terms of mg/kg of body weight per day.

In practice, the therapeutically effective amount may vary depending on numerous factors associated with the patient, including age, weight, height, severity of the condition, administration technique, and other factors. The therapeutically effective amount administered to a patient may be determined by medical personnel taking into account the relevant circumstances.

The therapeutically effective amount may be determined or predicted from empirical evidence. Specific dosages may vary according to numerous factors and may be initially determined on the basis of experimentation.

The product may be administered as a single dose or as part of a dosage regimen. For a dosage regimen, the therapeutically effective amount is adjustable dose to dose to provide a desired therapeutic response.

Multiple doses may be administered at a predetermined time interval and subsequent doses may be proportionally reduced or increased, depending on the situation.

The dosage form may be coated with a seal coating. Examples of seal coating materials include, but are not limited to, hydroxypropyl cellulose, hypromellose, and polyvinyl alcohol.

An example of a method for making a melatonin composition is now described.

The ingredients from which the dosage form is composed are combined in a container and mixed together in dry form without including a solvent such as water. Melatonin powder having a median melatonin particle size of 5 μm to 40 μm, carboxylic acid powder, and a powder of a hydrogel-forming polymer are mixed to form a dry granulation having a substantially uniform distribution of melatonin powder, carboxylic acid powder and powder of the hydrogel-forming polymer throughout.

The dry granulation is further processed using a compaction technique such as slugging and/or roller compaction. A roller compaction device squeezes the dry granulation through counter-rotating rollers to form a compressed sheet of the dry granulation. The sheet is broken into flakes and the flakes are milled to the desired size granules. The granules are then compressed together into the final dosage form.

Notably, the composition is prepared without solubilizing the melatonin before combining with the excipients so that the melatonin in the final dosage form ready to be administered to the patient is in its micronized crystalline form. There is also no need to perform a milling step on melatonin and the excipients because the milling step can cause lose and/or degradation of melatonin.

Tablets and caplets may be prepared using conventional tableting techniques such as dry granulation and compaction. The dry granulation may be compressed or compacted into a final dosage form.

Capsules may be prepared using different techniques. For example, granules produced by dry granulating the ingredients may be loaded into a capsule, such as a gelatin capsule.

Alternatively, the capsules or sachets may be loaded with individual multiparticulates having a diameter of from about 0.5 mm to about 4 mm or from about 0.5 mm to about 3 mm. The individual particulates may include any of the coatings discussed here.

Wet blending and wet granulation are to be avoided when preparing the dosage form. It is preferred that the materials used to prepare the dosage form remain as dry as reasonably possible during processing. This allows the melatonin particles to be in direct physical contact with the acid particles in the dosage form. As mentioned above, adding water during the blending and granulation can cause melatonin to deaminate and can reduce the content uniformity of the dosage form.

The composition in any of the forms described above may be used to treat one or more conditions such as insomnia, melatonin deficiency, a sleep disorder, a circadian rhythm disorder, or the like. The composition may also be used to treat any condition for which melatonin would be therapeutically effective.

A patient in need of treatment may be treated by administering the composition described above to the patient. The product may be administered orally. The patient may be a human or animal patient.

### EXAMPLES

This section describes a few more specific examples of the composition. These examples are presented for illustrative purposes and are not intended to limit the scope of what is claimed or the scope of possible example embodiments.

### Example 1

In this prospective example, the composition includes micronized melatonin in the dosage form. The composition is prepared without milling the melatonin and by dry blending and compressing micronized melatonin and the excipients into a small (0.25 gram-1.0 gram) oral dosage form such as pill. A list of components and ranges for ingredients in the composition are in Table 1 below. The micronized melatonin has a median particle size of 5 μm to 40 μm. Ranges are presented in terms of mg (milligrams) and as % w/w (weight percent). The remaining portions of the pill may be composed of excipients used to prepare the final dosage form. Any combination of the ingredients in the % w/w listed below may be employed.

TABLE 1
Contents of an example of the dosage form

| Ingredient | Range (mg) | | | Range (% w/w) | | |
|---|---|---|---|---|---|---|
| Micronized | 0.1-20 | 1-10 | 1-10 | 0.04-8 | 0.4-4 | 0.04-8 |
| melatonin | | | | | | |
| Carboxylic | 30-100 | 45-85 | 60-75 | 12-40 | 18-34 | 24-30 |
| acid powder | | | | | | |
| Hydrogel- | 20-120 | 25-75 | 40-60 | 8-48 | 10-30 | 16-24 |
| forming | | | | | | |
| polymer | | | | | | |

### Example 2

In this prospective example, the composition includes micronized melatonin in the dosage form and the dosage form includes additional ingredients to form a 250 mg pill. The composition is prepared by dry blending and compressing micronized melatonin and the excipients into the dosage form. A list of components and ranges for ingredients in the composition are in Table 2 below. The micronized melatonin has a median particle size of 5 μm to 40 μm. Ranges are presented in terms of mg (milligrams) and as % w/w (weight percent). Any combination of the ingredients in the % w/w listed below may be employed.

TABLE 2
Contents of an example of the dosage form

| | | |
|---|---|---|
| Ingredient | Range (mg) | Range (% w/w) |

| | | | | |
|---|---|---|---|---|
| Micronized | 0.1-20 | 1-10 | 0.04-8 | 0.4-4 |
| melatonin | | | | |
| Citric acid | 30-100 | 60-75 | 12-40 | 24-30 |
| Binder (e.g. | 20-120 | 40-60 | 8-48 | 16-24 |
| HPMC) | | | | |
| Filler (e.g. | 50-200 | 70-100 | 20-80 | 28-40 |
| microcrystalline | | | | |
| cellulose) | | | | |
| Filler (e.g. | 5-50 | 10-30 | 2-20 | 4-12 |
| pregelatinized | | | | |
| starch) | | | | |
| Flow agent (e.g. | 1-20 | 2-6 | 0.4-8 | 0.8-3 |
| magnesium | | | | |
| stearate | | | | |
| Flow agent (e.g. | 1-25 | 8-18 | 0.4-10 | 3-7 |
| talc) | | | | |
| Anticaking agent | 0.5-4 | 0.5-3 | 0.2-1.6 | 0.2-1.2 |
| (e.g. colloidal | | | | |
| silicon dioxide) | | | | |
| Coloring agent | 1-15 | 4-6 | 0.4-6 | 1.6-2.4 |
| (pigments) | | | | |
| TOTAL | 250 | 250 | | |

This disclosure has described example embodiments, but not all possible embodiments of the compositions or associated methods. Where a particular feature is disclosed in the context of a particular embodiment, that feature can also be used, to the extent possible, in combination with and/or in the context of other embodiments. The compositions and related methods may be embodied in many different forms and should not be construed as limited to only the embodiments described here.

That which is claimed is:

1. A method of making a melatonin dosage form, the method comprising:
forming granules by dry granulating a melatonin powder having a median melatonin particle size of 5 μm to 40 μm, a carboxylic acid powder, and a powder of a hydrogel-forming polymer to form a dry granulation having a substantially uniform distribution of melatonin powder, carboxylic acid powder, and powder of the hydrogel-forming polymer; and
placing the granules into a dry orally ingestible pharmaceutical dosage form, the dosage form being adapted to absorb water upon ingestion and form a hydrogel including soluble melatonin and soluble carboxylic acid in the hydrogel, the carboxylic acid being in an amount sufficient to impart a pH of 4.4 or less to the hydrogel after ingestion.

2. The method of claim 1, wherein dry granulating is performed without including a liquid solvent.

3. The method of claim 1, wherein the dosage form is selected from a tablet, capsule, caplet, and multiparticulate.

4. The method of claim 1, wherein the carboxylic acid is citric acid.

5. The method of claim 1, wherein a particle size of the carboxylic acid is greater than the particle size of the melatonin.

6. The method of claim 1, further comprising compacting the granules by roller compaction and/or slugging after the forming step and prior to the placing step.

7. The method of claim 1, wherein the dosage form comprises 0.4% w/w to 8% w/w melatonin, 12% w/w to 40% w/w carboxylic acid, and 8% w/w to 48% w/w hydrogel-forming polymer.

8. The method of claim 1, wherein the dosage form comprises 0.4% w/w to 8% w/w melatonin, 24% w/w to 30% w/w carboxylic acid, and 16% w/w to 24% w/w hydrogel-forming polymer.

9. The method of claim 1, wherein the dosage form provides a sustained release of melatonin after ingestion for 3-10 hours regardless of the pH environment the dry orally ingestible pharmaceutical dosage form passes through.

10. The method of claim 1, wherein the melatonin powder and carboxylic acid powder are in direct physical contact in the granules.

11. A melatonin composition comprising:
a dry granulation including a melatonin powder with a median melatonin particle size of 5 μm to 40 μm, a carboxylic acid powder, and a powder of a hydrogel-forming polymer;
the dry granulation being combined with pharmaceutical excipients in a dry orally ingestible pharmaceutical dosage form, the dosage form being adapted to absorb water upon ingestion and form a hydrogel including soluble melatonin and soluble carboxylic acid in the hydrogel, the carboxylic acid being in an amount sufficient to impart a pH of 4.4 or less to the hydrogel after ingestion.

12. The composition of claim 11, wherein the dosage form is at least one of a tablet, capsule, and multiparticulate.

13. The composition of claim 11, wherein the carboxylic acid is citric acid.

14. The composition of claim 11, wherein a particle size of the carboxylic acid is greater than the particle size of the melatonin.

15. The composition of claim 11, wherein the dosage form comprises 0.4% w/w to 8% w/w melatonin, 12% w/w to 40% w/w carboxylic acid, and 8% w/w to 48% w/w hydrogel-forming polymer.

16. The composition of claim 11, wherein the dosage form comprises 0.4% w/w to 8% w/w melatonin, 24% w/w to 30% w/w carboxylic acid, and 16% w/w to 24% w/w hydrogel-forming polymer.

17. The composition of claim 11, wherein the dosage form provides a sustained release of melatonin after ingestion for 3-10 hours regardless of the pH environment the dosage form passes through.

18. The composition of claim 1, wherein the melatonin powder and carboxylic acid powder are in direct physical contact in the dry granulation.

19. A method of treatment comprising:
administering to a patient in need of melatonin therapy a therapeutically effective amount of a dry orally ingestible pharmaceutical dosage form having therein a dry granulation including a melatonin powder with a median melatonin particle size of 5 μm to 40 μm, a carboxylic acid powder, and a powder of a hydrogel-forming polymer;
the dry granulation being combined with pharmaceutical excipients in the dosage form, the dosage form being adapted to absorb water upon ingestion and form a hydrogel including soluble melatonin and soluble carboxylic acid in the hydrogel, the carboxylic acid being in an amount sufficient to impart a pH of 4.4 or less to the hydrogel after ingestion.

20. The method of claim 19, wherein the dosage form is selected from a tablet, capsule, caplet, and multiparticulate.

21. The method of claim 19, wherein the carboxylic acid is citric acid.

22. The method of claim 19, wherein a particle size of the carboxylic acid is greater than the particle size of the melatonin.

23. The method of claim 19, wherein the dosage form comprises 0.4% w/w to 8% w/w melatonin, 12% w/w to 40% w/w carboxylic acid, and 8% w/w to 48% w/w hydrogel-forming polymer.

24. The method of claim 19, wherein the dosage form comprises 0.4% w/w to 8% w/w melatonin, 24% w/w to 30% w/w carboxylic acid, and 16% w/w to 24% w/w hydrogel-forming polymer.

25. The method of claim 19, wherein the dosage form provides a sustained release of melatonin after ingestion for 3-10 hours regardless of the pH environment the dosage form passes through.

26. The method of claim 19, wherein the melatonin powder and carboxylic acid powder are in direct physical contact in the dry granulation.
